(19) United States
(12) Patent Application Publication
(10) Pub. No.: US 2014/0330283 A1
(43) Pub. Date: Nov. 6, 2014

(54) Adjustable Surgical Instruments

(71) Applicant: Ravi Nallakrishnan, Westmont, IL (US)

(72) Inventor: Ravi Nallakrishnan, Westmont, IL (US)

(21) Appl. No.: 12/788,323

(22) Filed: May 27, 2010

Related U.S. Application Data
(60) Provisional application No. 61/224,058, filed on Jul. 9, 2009.

(51) Int. Cl. A61F 9/007

(52) U.S. Cl. CPC A61F 9/00736
USPC 606/107

(57) ABSTRACT

Surgical instruments for ophthalmic surgery are modified to include a manually bendable portion to allow the position of the operative segment of the instrument to be adjusted during surgery without the use of adjusting tools. In one embodiment the bendable portion is formed by selectively annealing a site on the operative segment of the instrument. Commonly used instruments are then made more useful by allowing the surgeon to make adjustments during surgery to assure comfort and utility.

This application claims priority from U.S. Provisional Patent Application Ser. No. 61/224,058, filed 9 Jul. 2009, which is hereby incorporated herein, in its entirety, by reference.

This invention relates generally to surgical instruments and, more particularly, to instruments used in eye surgery which feature bendable or adjustable portions.

### BACKGROUND OF THE INVENTION

Various procedures performed during eye surgery require the use of specialized instruments often shaped and constructed to perform a specific task as part of the surgical procedure. Instruments such as hooks, speculums, markers, forceps, scissors, manipulators and other instruments have components shaped and sized to carry out specific tasks.

Selected examples of such instruments are marketed by ASICO, LLC. of Westmont, Ill. For example, the May Muscle Hook, No. AE-2125, is used to manipulate the muscles of the eye during surgery. The Lestor IOL Pusher (No. AE-2410) is a surgical spatula used for contacting and maneuvering an intraocular lens (IOL) once it has been inserted into the eye. The Kansas Linear Marker (No. AE-1525) is an ophthalmic marker used for placing reference marks on the sclera. The Manche Speculum (No. AE-1039) is a surgical instrument used to hold the eye open during surgical procedures. The Castroviejo-Colibri Forceps (No. AE-4053) is a surgical forceps used to grip or hold the cornea during surgery. The intraocular scissors (No. AE-5600) is an ophthalmic surgical scissor used to cut eye tissue. The Little No Hole Manipulator (AE-2234) is a surgical tool used to maneuver an IOL after insertion.

These instruments are illustrative of the type and variety of instruments commonly used in ophthalmic surgery where the operating field is often small and confined.

### BACKGROUND OF THE INVENTION

A degree of adjustability and convenience is provided to commonly-used surgical instruments by manufacturing the instruments with an annealed segment allowing the instrument to be bent at the site of the annealing. The selection of the annealed site varies with the type of instrument being used. This range of adjustment allows the use of a single instrument which may be adjusted during surgery as opposed to requiring the use of several instruments of the same type but with tips or portions formed at different angles to be used in different parts of the surgical procedure.

Surgical instruments having deformable, bendable or malleable features are represented in the prior art.

U.S. Pat. No. 7,112,208 (Morris et al) teaches and describes a compact suture punch with malleable needle consisting of a hand held instrument used to grip the tissue and a trocar for forcing a malleable or bendable needle through the tissue to be sutured. In other words, the needle is straight as it enters the instrument but curves as it is performing the suturing operation.

U.S. Pat. No. 7,147,651 (Morrison et al) teaches and describes a stiff tipped suture whereby an otherwise entirely flexible suture is provided with a stiff or non-bendable tip to make it easier to thread the suture through a surgical instrument and, thereafter, through tissue.

U.S. Pat. No. 5,818,571 (Johnson) teaches and describes an apparatus for bending a surgical instrument. This instrument is designed to allow the tip of a phacoemulsification needle to be bent to a convenient angle prior to insertion into the eye but is intended for use with otherwise non-bendable tips.

While the following describes a preferred embodiment or embodiments of the present invention, it is to be understood that this description is made by way of example only and is not intended to limit the scope of the present invention. It is expected that alterations and further modifications, as well as other and further applications of the principles of the present invention will occur to others skilled in the art to which the invention relates and, while differing from the foregoing, remain within the spirit and scope of the invention as herein described and claimed. Where means-plus-function clauses are used in the claims such language is intended to cover the structures described herein as performing the recited functions and not only structural equivalents but equivalent structures as well. For the purposes of the present disclosure, two structures that perform the same function within an environment described above may be equivalent structures.

### BRIEF DESCRIPTION OF THE DRAWINGS

These and further objects of the present invention will become more apparent upon consideration of the accompanying drawings which illustrate various aspects of the present invention in a manner which may or may not reflect actual proportions or dimensions.

FIG. 1 is a lateral perspective view of a prior art muscle hook;

FIG. 2 is a partial lateral perspective view of the instrument of FIG. 1 embodying certain principles of the present invention;

FIG. 3 is a lateral perspective view of a prior art IOL pusher;

FIG. 4 is a partial lateral sectional view of the instrument of FIG. 3, embodying certain principles of the present invention;

FIG. 5 is a lateral perspective view of a prior art linear marker;

FIG. 6 is an enlarged partial lateral view of the marker of FIG. 5 embodying certain principles of the present invention;

FIG. 7 is a perspective view of a prior art speculum;

FIG. 8 is a perspective view of the speculum of FIG. 7 embodying certain principles of the present invention;

FIG. 9 is a perspective view of a prior art forceps;

FIG. 10 is a perspective view of the forceps of FIG. 9 embodying certain principles of the present invention;

FIG. 11 is a perspective view of a prior art intraocular scissors;

FIG. 12 is an enlarged partial perspective view of the scissors of FIG. 11 embodying certain principles of the present invention;

FIG. 13 is a lateral perspective view of a surgical manipulator; and

FIG. 14 is an enlarged partial lateral perspective view of the manipulator of FIG. 13 embodying certain principles of the present invention.

### DETAILED DESCRIPTION OF THE DRAWINGS

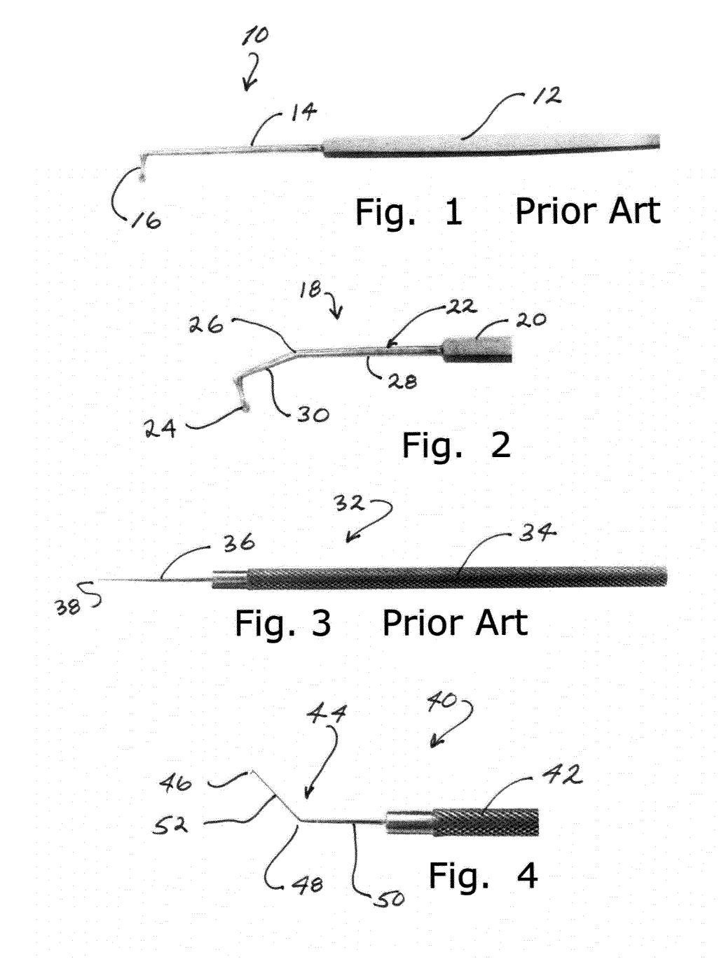

Referring now to FIG. 1, the numeral 10 identifies a prior art muscle hook having a handle 12 attached to a hook shaft 14 and a hook end 16. This instrument is generally of the type identified above as the May muscle hook (AE-2125). Shaft 14 of hook 10 is typically formed as a stiff, straight shaft which does not easily flex or bend.

Referring now to FIG. 2, the numeral 18 identifies a muscle hook generally of the type shown in FIG. 1 embodying certain principles of the present invention. Hook 18 has a handle 20 and a hook shaft 22 terminating in a hook end 24. At a point intermediate handle 20 and hook end 24, hook shaft 22 is annealed or otherwise treated to form a bend point 26. As seen in FIG. 2, bend point 26 divides hook shaft 22 into a proximal segment 28 and a distal segment 30. Shaft 22 can be bent at point 26 to angle distal segment 30 with respect to proximal segment 28 plus changing the angle of hook end 24.

While FIG. 2 shows distal segment 30 angled in a downward direction relative to proximal segment 28, it should also be understood that bend point 26 can be formed to allow distal segment 30 to bend laterally and upwardly as well. It is also expected that distal segment 30 may be bent in a number of orientations intermediate these positions.

Referring now to FIG. 3, the numeral 32 identifies a prior art IOL pusher having a pusher shaft 36 terminating in a pusher end 38. Pusher shaft 36 is typically formed as a rigid shaft that does not easily bend or flex.

Referring now to FIG. 4, the numeral 40 identifies an IOL pusher embodying certain principles of the present invention. Pusher 40 has a handle 42 to which a pusher shaft 44 is attached with shaft 44 terminating in a pusher tip 46. A bend point 48 is formed on shaft 44 intermediate handle 42 and tip 46 dividing shaft 44 into a proximal segment 50 and a distal segment 52. Bend point 48 is formed by annealing or other metal treating methods to allow distal segment 52 to be bent or angled with respect to proximal segment 50. As described above, this instrument can be manufactured to allow bending and thus orientation of distal shaft segment 52 to proximal shaft segment 50 in lateral, horizontal, or other selected intermediate positions as desired.

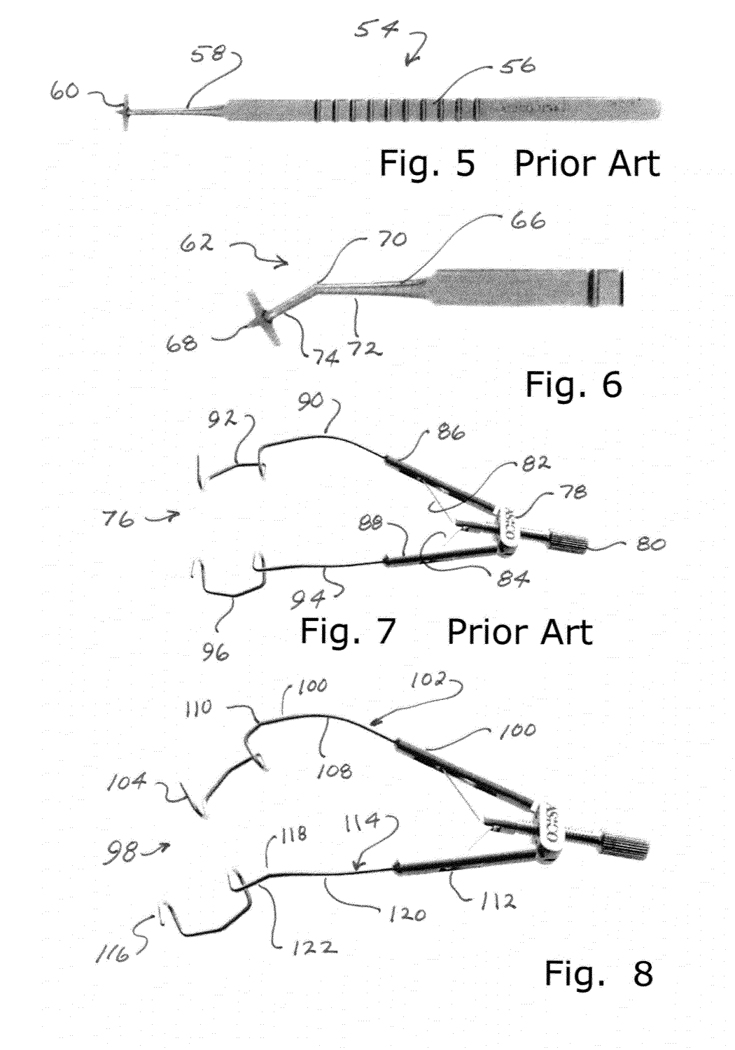

Referring now to FIG. 5, the numeral 54 identifies a prior art linear marker having handle 56 to which is attached a marker shaft 58. Marker shaft 58 terminates at a marker tip 60 adapted to place reference marks at selected positions on the sclera of an eye.

Referring now to FIG. 6, the numeral 62 identifies a linear marker embodying certain principles of the present invention. Marker 62 has a handle 64 to which is attached a shaft 66. Shaft 66 terminates in a marker tip 68. As seen in FIG. 6, a bend point 70 is formed on shaft 66 intermediate handle 64 and tip 68, dividing shaft 66 into a proximal segment 72 and a distal segment 74. Bend point 70 is formed by annealing or other metal treating methods and allows a distal segment 74 to be angled or adjusted with respect to proximal segment 72. As described above, this instrument can be manufactured to allow bending and thus orientation of distal segment 74 to proximal segment 72 in lateral, horizontal, or other selected intermediate positions as desired.

Referring now to FIG. 7, the numeral 76 identifies a prior art speculum having a yoke 78 with which an adjusting handle 80 is associated. Handle 80 has a first link 82 and a second link 84 slidably received, respectively, by yoke arms 86 and 88. First speculum arm 90 extends from yoke 86 and terminates in a first speculum hook 92. In like fashion, a second speculum arm 94 extends from yoke 88 and terminates in a second speculum hook 96. Both hooks 92 and 96 are sized and shaped to engage the external parts of the eye. Adjusting handle 80 forces yokes 86, 88 either towards each other or away from each other thereby changing the distance between hooks 92, 96.

Referring now to FIG. 8 a speculum 98 is shown embodying certain principles of the present invention. As seen in FIG. 8, first speculum arm 100 has a bend point 102 formed therealong intermediate speculum yoke 104 and speculum hook 106. First speculum arm 100 is thus divided into a proximal segment 106 and a distal segment 108. In like fashion, second speculum arm 110 extends from second yoke 112 and terminates in a second hook 114. A bend point 116 is formed along second speculum arm 110 intermediate yoke 112 and hook 114 thereby dividing second arm 110 into a first proximal segment 118 and a second distal segment 120. As seen in FIG. 8, the presence of bend points 102, 116 allows the movement of distal segments 108, 120 with respect to proximal segments 106, 118. In this manner, the relative positions of hooks 106, 114 may be changed without changing the shape of said hooks.

As described above, this instrument can be manufactured to allow bending and thus orientation of distal segments 108, 120 respectively, with respect to proximal segments 106, 118 respectively, in lateral, horizontal, or other selected intermediate positions as desired.

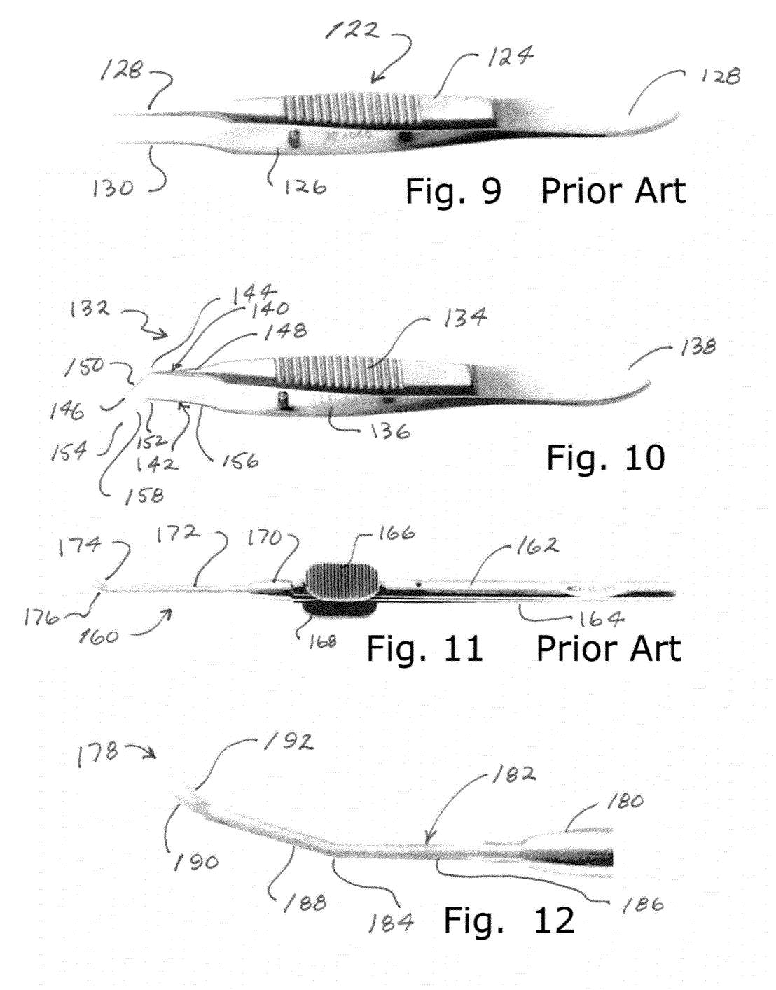

Referring now to FIG. 9, the numeral 122 identifies a prior art forceps having a first handle 124 and a second handle 126 joined at an end 128. Forceps 122 is formed from spring steel or similar material such that in its normal unstressed position, handles 124, 126 are urged apart from each other. When squeezed together, handles 124, 126 may be moved to engage each other.

Handle 124 terminates in a forceps tip 128 while handle 126 terminates in a forceps tip 130. When handles 124, 126 are squeezed together, tips 128, 130 perform a gripping action.

Referring now to FIG. 10, the numeral 132 identifies a forceps embodying certain principles of the present invention. Handles 134, 136 are joined at hinge 138 as described above in connection with FIG. 9. Handle 134 terminates in a tip 140 while handle 136 terminates in a tip 142. A bend point 144 is formed on tip 140 intermediate tip end 146 and handle 134. This divides tip 140 into a proximal segment 148 and a distal segment 150. In like fashion, second tip 142 has a bend point 152 positioned intermediate tip end 154 and handle 136. Tip 142 is thus divided into a proximal segment 156 and a distal segment 158.

In the embodiment shown, distal segments 150, 158 may be bent, respectively, in relation to proximal segments 148, 150, thereby allowing tips 140, 142 to take on differing configurations as the surgeon may require.

As described above, this instrument can be manufactured to allow bending and thus orientation of distal segments 150, 158, respectively, to proximal segments 148, 156, respectively, in lateral, horizontal, or other selected intermediate positions as desired.

Referring now to FIG. 11, the numeral 160 identifies a prior art intraocular scissors having a pair of opposed handles 162, 164 which are spring biased to remain apart from one another and are compressed towards each other by use of finger pads 166, 168. A nose piece 170 has extending therefrom a tube 172 within which a pair of spring steel halves 174, 176 are placed, terminating in a pair of scissors blades. Scissors 160 is of the type wherein the pressing of finger pads 166, 168 toward each other compresses handles 162, 164 and draws steel halves 174, 176 into tube 172 thereby bringing the blade ends together and cause the cutting action of the scissors.

Referring now to FIG. 12, the numeral 178 identifies a scissors construction embodying certain principles of the present invention. As seen, nose 180 forms an attachment point for tube 182. Blade halves 190, 192 extend from tube 182. A bend point 184 is formed on tube 182 intermediate nose 180 and blade tips 190, 192 thereby dividing tube 182 into a proximal portion 186 and a distal portion 188. Bend point 184 allows proximal portion 186 and distal portion 188 to be angled with respect to each other. In one embodiment of this arrangement is to have the bending of tube 182 be accomplished in a direction parallel to the direction in which the blade halves 190, 192 extend through tube 182 thereby allowing blade halves 190, 192 to be positioned slightly without interfering with the scissoring action of scissor 178. As described above, other orientations are also possible, depending upon the nature of the annealing performed.

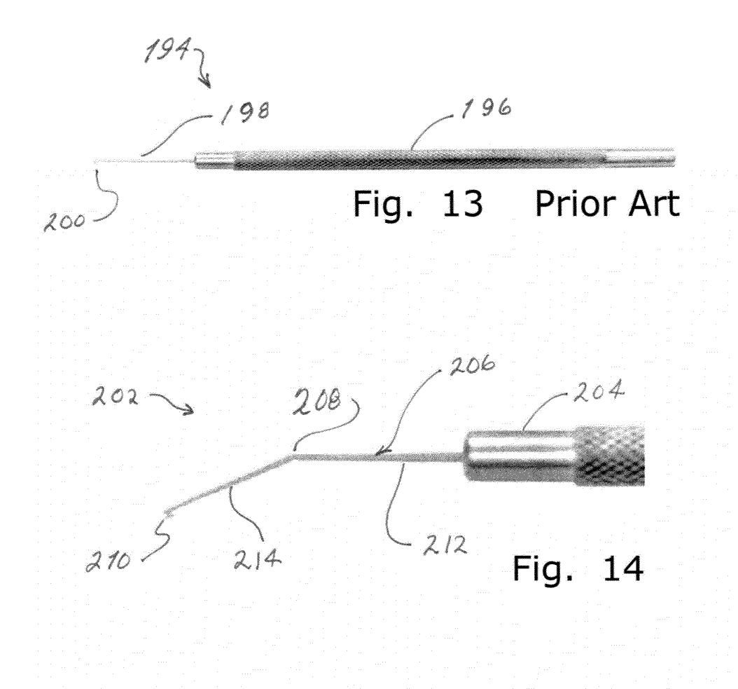

Referring now to FIG. 13, a prior art manipulator 194 is shown having a handle 196 from which a shaft 198 extends, ending in a manipulator tip 200.

Referring now to FIG. 14, the numeral 202 identifies a manipulator embodying certain principles of the present invention.

Tube 206 is secured to handle 204 and terminates in a tip 210. A bend point 208 is formed on shaft 206 intermediate handle 204 and tip 210 thereby forming a proximal portion 212 and a distal portion 214 of shaft 206.

In use, bend point 208 allows distal portion 214 to be angled with respect to proximal portion 212, thereby making manipulator 202 adjustable. As described above, this instrument can be manufactured to allow bending and thus orientation of distal portion 214 to proximal portion 212 in lateral, horizontal, or other selected intermediate positions as desired.

While the examples used herein relate to instruments for ophthalmic surgery the principles of the present invention can readily be applied to instruments and tools used in other disciplines as well.

What is claimed is:

1. A surgical instrument adapted for use in ophthalmic surgery, said instrument comprising:
a handle;
means for performing a surgical procedure extending from said handle,
said surgical procedure means having at least one instrument segment adapted to carry out said surgical procedure,
said at least one instrument segment terminating at an instrument end; and
means formed on said at least one instrument segment intermediate to allow said segment to be manually bent, whereby the position of said instrument segment extending from said bending means to said instrument end may be adjusted with respect to that portion of said instrument segment extending from said bending means to said handle.

2. The apparatus as recited in claim 1 wherein said bending means comprises a portion of said instrument segment that has been heat treated to make said segment portion bendable using manual force.

3. The apparatus as recited in claim 2 wherein said surgical instrument is a muscle hook having a shaft extending from said handle and terminating at a hook end,
said bending means formed intermediate said handle and said hook end.

4. The apparatus as recited in claim 2 wherein said surgical instrument is an IOL pusher having a shaft extending from said handle and terminating at a shaft tip,
said bending means formed intermediate said handle and said shaft tip.

5. The apparatus as recited in claim 2 wherein said surgical instrument is a linear marker having a shaft extending from said handle and terminating at plurality of marking tabs,
said bending means formed intermediate said handle and said marking tabs.

6. The apparatus as recited in claim 2 wherein said surgical instrument is a speculum having a yoke with a pair of speculum arms extending therefrom, each said arm terminating at a speculum loop,
said bending means formed intermediate said handle and at least one of said speculum loops.

7. The apparatus as recited in claim 2 wherein said surgical instrument is a forceps having a pair of forceps arms extending from said handle,
each said forceps arm terminating at a forceps tip,
said bending means formed on at least one said forceps arm intermediate said handle and said forceps tip.

8. The apparatus as recited in claim 7 wherein one said bending means is formed on each said forceps arm,
said bending means being aligned when said forceps arms are positioned together.

9. The apparatus as recited in claim 2 wherein said surgical instrument is a scissors having a pair of opposed cutting strips positioned within a hollow shaft extending from said handle and terminating at a pair of scissor jaws,
said bending means formed intermediate on said hollow tube intermediate said handle and said scissor jaws.
